(19) United States
(12) Patent Application Publication
(10) Pub. No.: US 2025/0176804 A1
(43) Pub. Date: Jun. 5, 2025

(54) ENDOSCOPE HOLDING DEVICE, ENDOSCOPIC SURGERY SYSTEM, AND CONTROL METHOD

(71) Applicant: SONY GROUP CORPORATION, TOKYO (JP)

(72) Inventors: KEI TOMATSU, TOKYO (JP); YOHEI KURODA, TOKYO (JP); JUN ARAI, TOKYO (JP)

(21) Appl. No.: 18/843,839

(22) PCT Filed: Jan. 19, 2023
(86) PCT No.: PCT/JP2023/001475

§ 371 Date: Sep. 4, 2024

(30) Foreign Application Priority Data
Mar. 15, 2022 (JP) 2022-040681

(51) Int. Cl. A61B 1/00; A61B 1/06

(52) U.S. Cl. CPC A61B 1/00149; A61B 1/0016; A61B 1/00179; A61B 1/0646

(57) ABSTRACT

The present disclosure provides an endoscope holding device, an endoscopic surgery system, and a control method capable of curbing an increase in size.

According to the present disclosure, an endoscope holding device includes: a housing that accommodates a relay optical system and is disposed in a robot arm; a first rotation unit that is disposed in the housing and rotates an endoscope with respect to an optical axis of the relay optical system, the endoscope being fixed such that light emitted from the endoscope enters the relay optical system, and a second rotation unit that is disposed in the housing and rotates an imaging device with respect to the optical axis of the relay optical system, the imaging device being fixed such that light emitted from the relay optical system enters the imaging device.

### TECHNICAL FIELD

The present disclosure relates to an endoscope holding device, an endoscopic surgery system, and a control method.

### BACKGROUND ART

Recently, an oblique-viewing endoscope is used as a rigid endoscope to be inserted into a human body. In addition, a forward-viewing endoscope in which polarization processing is performed is generally known.

### CITATION LIST

### Patent Document

Patent Document 1: Japanese Patent Application Laid-Open No. 7-234365

### SUMMARY OF THE INVENTION

### Problems to be Solved by the Invention

However, since the perspective rotation by the oblique-viewing endoscope turns with respect to the scope axis, it is difficult to intuitively understand the field of view after the rotation as compared with simple movement in the horizontal direction or the vertical direction. In addition, in the forward-viewing endoscope in which polarization processing is performed, the polarization deviates when the rigid endoscope is rotated with respect to the scope axis. For this reason, it has been attempted to capture a target image by controlling rotation of each of the rigid endoscope and a camera head portion by a robot arm.

However, if the rigid endoscope and the camera head portion are to be rotatably held by the robot arm, the endoscope holding device, which is the distal end portion of the robot arm, becomes large, and there is a possibility that the endoscope holding device interferes with the operator's arm and hinders surgery.

Therefore, the present disclosure provides an endoscope holding device, an endoscopic surgery system, and a control method capable of curbing an increase in size.

### Solutions to Problems

In order to solve the above problem, the present disclosure provides an endoscope holding device including: a housing that accommodates a relay optical system and is disposed in a robot arm; a first rotation unit that is disposed in the housing and rotates an endoscope with respect to an optical axis of the relay optical system, the endoscope being fixed such that light emitted from the endoscope enters the relay optical system; and a second rotation unit that is disposed in the housing and rotates an imaging device with respect to the optical axis of the relay optical system, the imaging device being fixed such that light emitted from the relay optical system enters the imaging device.

A first attachment portion that detachably attaches the endoscope to the first rotation unit may be further provided.

The first attachment portion may fix the endoscope such that a first optical axis at a subsequent stage of the endoscope coincides with a second optical axis of the relay optical system.

A first rotation angle acquisition unit that acquires a rotation angle of the endoscope rotating around the first optical axis may be further provided.

A first electric motor unit that rotates the endoscope around the first optical axis may be further provided.

The second optical axis and a third optical axis of an optical system of the imaging device may be fixed to coincide with each other.

The second rotation unit may rotate the imaging device around the third optical axis on the basis of information of a rotation angle of the endoscope acquired by the first rotation angle acquisition unit.

A second attachment portion that detachably attaches the imaging device to the second rotation unit may be further provided.

The relay optical system and an optical system of the imaging device may be integrally formed.

A second electric motor unit that rotates the imaging device around the third optical axis may be further provided.

The second electric motor unit may rotate the imaging device around the third optical axis on the basis of information of a rotation angle of the endoscope.

The endoscope may be an oblique-viewing endoscope.

The second electric motor unit may rotate the imaging device with respect to the third optical axis on the basis of information of a rotation angle of the endoscope such that the imaging device maintains a predetermined rotation angle with respect to the third optical axis.

The endoscope may be a forward-viewing endoscope.

A first polarizing filter may be disposed in an optical system of the endoscope, and a second polarizing filter may be disposed in an optical system of the imaging device, and
the second electric motor unit may rotate the imaging device with respect to the third optical axis on the basis of information of a rotation angle of the endoscope such that the first polarizing filter and the second polarizing filter maintain a predetermined rotation angle.

In order to solve the above problem, the present disclosure provides an endoscopic surgery system including:
the endoscope holding device described above;
a mark generation unit that generates a mark indicating at least one of a predetermined direction with respect to a third optical axis of the imaging device and a rotation direction by using information of a rotation angle of the endoscope acquired by the first rotation angle acquisition unit; and
a control unit that causes a display device to display an image captured by the imaging device with the mark superimposed on the image.

An acceleration sensor may be disposed in the imaging device, and
the mark generation unit may generate a mark indicating at least one of a predetermined direction with respect to the third optical axis and a rotation direction by also using information of the acceleration sensor.

The mark generation unit may generate a mark indicating information of the rotation direction and an angle to be rotated.

A support arm device that supports the robot arm including the endoscope holding device at an end portion may be further provided.

In order to solve the above problem, the present disclosure provides a control method of an endoscope holding device including
a first rotation step of rotating an endoscope with respect to an optical axis of a relay optical system, the endoscope being fixed to one end of a housing accommodating the relay optical system such that light emitted from the endoscope enters the relay optical system, and
a second rotation step of rotating an imaging device with respect to the optical axis of the relay optical system according to a rotation angle of the endoscope, the imaging device being fixed to the other end of the housing such that light emitted from the relay optical system enters the imaging device.

### BRIEF DESCRIPTION OF DRAWINGS

FIG. 1 is a diagram illustrating an example of a schematic configuration of an endoscopic surgery system to which a technology according to the present disclosure can be applied.

FIG. 2 is a schematic diagram illustrating a configuration in which a holding unit is provided at a distal end of an arm unit.

FIG. 3 is a diagram illustrating a configuration example of an endoscope unit.

FIG. 4 is a schematic external view illustrating an endoscope of a first comparative example.

FIG. 5 is a schematic external view illustrating an endoscope of a second comparative example.

FIG. 6 is a schematic external view illustrating the endoscope unit according to the present embodiment.

FIG. 7 is a block diagram illustrating an example of functional components of an endoscope unit, a CCU, and an arm control device.

FIG. 8 is a block diagram of a configuration example related to rotation control with respect to a holding unit portion in a control unit.

FIG. 9 is a schematic diagram illustrating an appearance of an oblique-viewing endoscope.

FIG. 10 is a schematic diagram illustrating how a visual field displayed on a display device changes due to perspective rotation.

FIG. 11 is a flowchart illustrating an example of rotation control of the oblique-viewing endoscope and a camera head.

FIG. 12 is a diagram illustrating a configuration example of an endoscope unit according to a second embodiment.

FIG. 13 is a block diagram of a configuration example related to rotation control with respect to a holding unit portion according to the second embodiment.

FIG. 14 is a diagram for describing an outline of the present embodiment.

FIG. 15 is a flowchart illustrating a control example according to the second embodiment.

FIG. 16 is a diagram for describing an outline of Modification 1 of the second embodiment.

FIG. 17 is a flowchart illustrating a control example according to Modification 1 of the second embodiment.

FIG. 18 is a diagram illustrating a configuration example of an endoscope unit according to Modification 2 of the second embodiment.

FIG. 19 is a diagram illustrating a configuration example of a camera head according to Modification 3 of the second embodiment.

FIG. 20 is a diagram illustrating a configuration example of an endoscope unit according to a third embodiment.

### MODE FOR CARRYING OUT THE INVENTION

Hereinafter, preferred embodiments of the present disclosure will be described in detail with reference to the accompanying drawings. Note that in the present specification and the drawings, components having substantially the same functional configuration are denoted by the same reference signs, and redundant explanations are omitted.

### First Embodiment

### 1. Basic Configuration>>

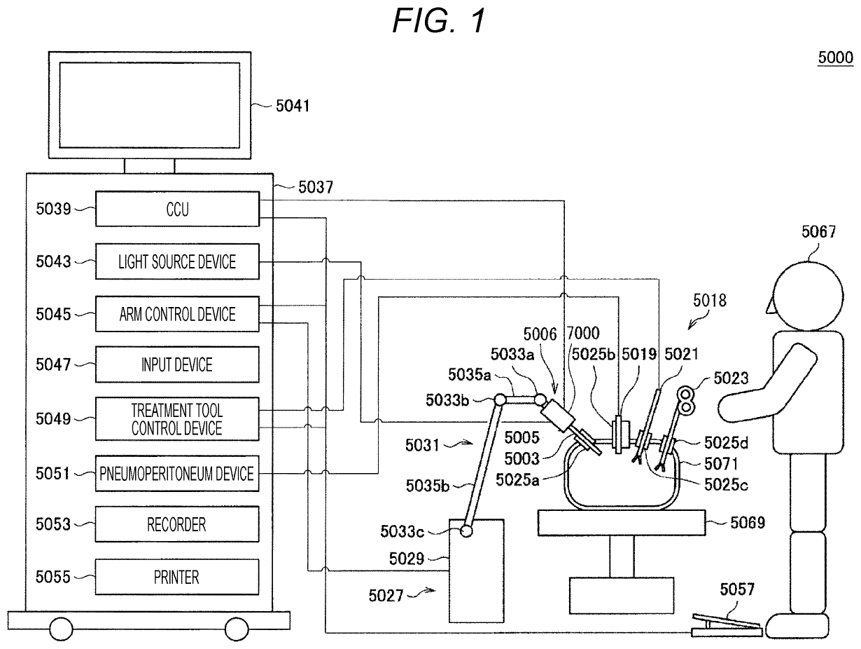

First, a configuration example of an endoscopic surgery system will be described with reference to FIG. 1.

### <1.1. Configuration Example of Endoscope System>

FIG. 1 is a diagram illustrating an example of a schematic configuration of an endoscopic surgery system 5000 to which the technology according to the present disclosure can be applied. In FIG. 1, a state is depicted in which an operator (surgeon) 5067 is using the endoscopic surgery system 5000 to perform surgery on a patient 5071 on a patient bed 5069. As illustrated, the endoscopic surgery system 5000 includes an endoscope unit 5006, other surgical tools 5018, a support arm device 5027 that supports the endoscope unit 5006, and a cart 5037 on which various devices for endoscopic surgery are mounted.

In endoscopic surgery, instead of cutting the abdominal wall to open the abdomen, a plurality of tubular opening devices called trocars 5025a to 5025d is punctured into the abdominal wall. Then, a lens barrel 5003 of the endoscope unit 5006 and the other surgical tools 5018 are inserted into the body cavity of the patient 5071 from the trocars 5025a to 5025d. In the illustrated example, a pneumoperitoneum tube 5019, an energy treatment tool 5021, and forceps 5023 are inserted into the body cavity of the patient 5071 as the other surgical tools 5018. Furthermore, the energy treatment tool 5021 is a treatment tool that performs incision and exfoliation of tissue, sealing of a blood vessel, or the like by high-frequency current and ultrasonic vibration. Note, however, that the illustrated surgical tools 5018 are merely examples, and various surgical tools generally used in endoscopic surgery, such as tweezers and a retractor, may be used as the surgical tools 5018.

An image of a surgical site in the body cavity of the patient 5071 captured by the endoscope unit 5006 is displayed on a display device 5041. The operator 5067 performs a treatment such as resection of an affected site, for example, by using the energy treatment tool 5021 and the forceps 5023 while viewing the image of the surgical site displayed on the display device 5041 in real time. Note that although not illustrated, the pneumoperitoneum tube 5019, the energy treatment tool 5021, and the forceps 5023 are supported by the operator 5067, an assistant, or the like during the surgery.

### (Support Arm Device)

The support arm device 5027 includes an arm 5031 extending from a base 5029. In the illustrated example, the arm 5031 includes joints 5033a, 5033b, and 5033c, and links 5035a and 5035b, and is driven under the control of an arm control device 5045. The arm 5031 supports the endoscope unit 5006 and controls its position and posture. As a result, stable fixation of the position of the endoscope unit 5006 can be achieved.

### (Endoscope Unit)

The endoscope unit 5006 according to the present embodiment includes the lens barrel 5003 in which a region of a predetermined length from the distal end is inserted into the body cavity of the patient 5071, a holding unit portion 7000 connected to the proximal end of the lens barrel 5003, and a camera head 5005 connected to the proximal end of the holding unit portion 7000. In the illustrated example, the endoscope unit 5006 configured as a so-called rigid scope including the rigid lens barrel 5003 is illustrated, but the endoscope unit 5006 may be configured as a so-called flexible scope including the flexible lens barrel 5003.

At the distal end of the lens barrel 5003, an opening into which an objective lens is fitted is provided. A light source device 5043 is connected to the endoscope unit 5006, and light generated by the light source device 5043 is guided to the distal end of the lens barrel 5003 by a light guide extending in the lens barrel 5003 and is emitted to an observation target in the body cavity of the patient 5071 through the objective lens. Note that the lens barrel 5003 according to the present embodiment is an oblique-viewing endoscope and corresponds to an endoscope.

An optical system and an imaging element are provided inside the camera head 5005, and reflected light (observation light) from the observation target is condensed on the imaging element by the optical system. The observation light is photoelectrically converted by the imaging element, and an electric signal corresponding to the observation light, that is, an image signal corresponding to an observation image is generated. The image signal is transmitted as RAW data to a camera control unit (CCU) 5039. Note that the camera head 5005 has a function of adjusting magnification and focal length by appropriately driving the optical system thereof.

Note that the camera head 5005 may be provided with a plurality of imaging elements in order to support, for example, stereoscopic viewing (3D display) and the like. In this case, a plurality of relay optical systems is provided inside the lens barrel 5003 in order to guide the observation light to each of the plurality of imaging elements. Further, details of the endoscope unit 5006 according to the present embodiment will be described later.

### (Various Devices Mounted on Cart)

The CCU 5039 includes a central processing unit (CPU), a graphics processing unit (GPU), or the like and integrally controls operation of the endoscope unit 5006 and the display device 5041. Specifically, the CCU 5039 applies, to the image signal received from the camera head 5005, various types of image processing for displaying an image based on the image signal, such as development processing (demosaic processing) and the like. The CCU 5039 provides the image signal subjected to the image processing to the display device 5041. Furthermore, the CCU 5039 transmits a control signal to the camera head 5005, and controls driving of the camera head 5005. The control signal can include information regarding imaging conditions such as magnification and focal length.

The display device 5041 displays an image based on the image signal subjected to the image processing by the CCU 5039 under the control of the CCU 5039. In a case where, for example, the endoscope unit 5006 is compatible with high resolution imaging such as 4K (3840 pixels (horizontal)×2160 pixels (vertical)) or 8K (7680 pixels (horizontal)×4320 pixels (vertical)), and/or in a case where the endoscope unit 5006 is compatible with 3D display, a display device which can achieve high resolution display and/or 3D display to be adapted to both cases may be used as the display device 5041. In a case where the display device 5041 is compatible with high resolution imaging such as 4K or 8K, more immersive feeling can be obtained by using a display device 5041 having a size equal to or larger than 55 inches. Furthermore, a plurality of display devices 5041 having different resolutions and sizes may be provided depending on the application.

The light source device 5043 includes a light source such as a light emitting diode (LED), for example, and supplies irradiation light for imaging an operation site to the endoscope unit 5006.

The arm control device 5045 includes a processor such as a CPU and the like, for example, and operates according to a predetermined program to control driving of the arm 5031 of the support arm device 5027 according to a predetermined control method.

The input device 5047 is an input interface for the endoscopic surgery system 5000. A user can input various types of information and instructions to the endoscopic surgery system 5000 via the input device 5047. For example, the user inputs various types of information regarding the surgery such as physical information of the patient, and information regarding a surgical procedure via the input device 5047. Furthermore, for example, the user inputs an instruction to drive the arm 5031, an instruction to change the imaging condition by the endoscope unit 5006 (type of irradiation light, magnification, focal length, and the like), an instruction to drive the energy treatment tool 5021, and the like via the input device 5047.

The type of the input device 5047 is not limited, and the input device 5047 may be various known input devices. As the input device 5047, for example, a mouse, a keyboard, a touch panel, a switch, a foot switch 5057, and/or a lever and the like can be applied. In a case where a touch panel is used as the input device 5047, the touch panel may be provided on a display surface of the display device 5041.

Alternatively, the input device 5047 is a device worn by the user, such as an eyeglass-type wearable device, a head mounted display (HMD), and the like, for example, and various inputs are performed in accordance with the user's gesture and line-of-sight detected by these devices. Furthermore, the input device 5047 includes a camera capable of detecting movement of the user, and various inputs are performed in accordance with the user's gesture and line-of-sight detected from a video captured by the camera. Moreover, the input device 5047 includes a microphone capable of collecting the user's voice, and various inputs are performed by voice via the microphone. As described above, the input device 5047 is enabled to input various information without contact, whereby in particular the user (for example, the operator 5067) belonging to a clean area can operate a device belonging to an unclean area without contact. Furthermore, since the user can operate the device without releasing the user's hand from the surgical tool being held, the convenience of the user is improved.

A treatment tool control device 5049 controls driving of the energy treatment tool 5021 for cauterization and incision of tissue, sealing of a blood vessel, or the like. A pneumoperitoneum device 5051 feeds gas into the body cavity of the patient 5071 through the pneumoperitoneum tube 5019 to inflate the body cavity in order to secure the field of view of the endoscope unit 5006 and secure the operation space of the surgeon. A recorder 5053 is a device capable of recording various types of information regarding surgery. A printer 5055 is a device capable of printing various types of information regarding surgery in various formats such as text, image, or graph.

### (Support Arm Device)

The support arm device 5027 includes the base 5029 as a base, and the arm 5031 extending from the base 5029. In the illustrated example, the arm 5031 includes the plurality of joints 5033a, 5033b, and 5033c, and the plurality of links 5035a and 5035b connected together by the joint 5033b, but in FIG. 1, for simplicity, the configuration of the arm 5031 is illustrated in a simplified manner. Actually, shapes, the numbers, and arrangements of the joints 5033a to 5033c and the links 5035a and 5035b, directions of rotational axes of the joints 5033a to 5033c, and the like can be appropriately set so that the arm 5031 has a desired degree of freedom. For example, the arm 5031 can suitably have six or more degrees of freedom. As a result, since it becomes possible to freely move the endoscope unit 5006 within a movable range of the arm 5031, the lens barrel 5003 of the endoscope unit 5006 can be inserted into the body cavity of the patient 5071 from a desired direction.

Each of the joints 5033a to 5033c is provided with an actuator, and each of the joints 5033a to 5033c is rotatable around a predetermined rotational axis by driving of the actuator. The driving of the actuator is controlled by the arm control device 5045, whereby a rotation angle of each of the joints 5033a to 5033c is controlled, and the driving of the arm 5031 is controlled. As a result, the control of the position and posture of the endoscope unit 5006 can be achieved. At this time, the arm control device 5045 can control the driving of the arm 5031 by various known control methods such as force control or position control.

For example, the operator 5067 appropriately performs an operation input via the input device 5047 (including the foot switch 5057), whereby the driving of the arm 5031 may be appropriately controlled by the arm control device 5045 in accordance with the operation input, and the position and posture of the endoscope unit 5006 may be controlled. With this control, after the endoscope unit 5006 provided at the distal end of the arm 5031 is moved from an arbitrary position to an arbitrary position, the endoscope unit 5006 can be fixedly supported at the position after the movement. Note that the arm 5031 may be operated by a so-called master-slave method. In this case, the arm 5031 can be remotely operated by the user via the input device 5047 installed at a place away from an operating room.

Furthermore, in a case where the force control is applied, the arm control device 5045 may perform so-called power assist control to receive an external force from the user and drive the actuators of the joints 5033a to 5033c so that the arm 5031 moves smoothly according to the external force. As a result, when the user moves the arm 5031 while directly touching the arm 5031, the arm 5031 can be moved with a relatively light force Thus, the endoscope unit 5006 can be moved more intuitively and by a simpler operation, and the convenience of the user can be improved.

Here, generally, in endoscopic surgery, the endoscope unit 5006 is supported by a doctor called a scopist. On the other hand, by using the support arm device 5027, the position of the endoscope unit 5006 can be more reliably fixed without manual operation, so that the image of the surgical site can be stably obtained and the surgery can be performed smoothly.

Note that the arm control device 5045 does not necessarily have to be provided on the cart 5037. Furthermore, the arm control device 5045 does not necessarily have to be one device. For example, the arm control device 5045 may be provided on each of the joints 5033a to 5033c of the arm 5031 of the support arm device 5027, and a plurality of arm control devices 5045 may cooperate with each other to achieve driving control of the arm 5031.

### (Light Source Device)

The light source device 5043 supplies irradiation light for imaging the surgical site the like to the endoscope unit 5006. The light source device 5043 includes, for example, a white light source including an LED, a laser light source, or a combination thereof. At this time, in a case where the white light source includes a combination of RGB laser light sources, the output intensity and the output timing of each color (each wavelength) can be controlled with high accuracy, whereby the light source device 5043 can adjust the white balance of the captured image. Furthermore, in this case, by irradiating the observation target with laser light from each of the R, G, and B laser light sources by time division and controlling driving of the imaging element of the camera head 5005 in synchronism with the irradiation timing, it is possible to capture images corresponding to R, G, and B by time division. According to this method, a color image can be obtained even if color filters are not provided for the imaging element.

Furthermore, driving of the light source device 5043 may be controlled such that the intensity of the light to be output is changed every predetermined time. By controlling the driving of the imaging element of the camera head 5005 in synchronization with the timing of change of light intensity to obtain images by time division and combining the images, an image of a high dynamic range without so-called black defect and halation can be generated.

Furthermore, the light source device 5043 may be capable of to supplying light of a predetermined wavelength band corresponding to special light observation. In special light observation, for example, by utilizing the wavelength dependency of absorption of light in a body tissue to irradiate light of a narrower band than that of irradiation light (namely, white light) at ordinary observation, so-called narrow band observation (narrow band imaging) of imaging a predetermined tissue such as a blood vessel in a mucosal surface layer or the like with high contrast is performed. Alternatively, in special light observation, fluorescence observation of obtaining an image by fluorescence generated by emitting excitation light may be performed. In fluorescence observation, it is possible to irradiate a body tissue with excitation light to observe the fluorescence from the body tissue (autofluorescence observation), or to locally inject a reagent such as indocyanine green (ICG) into a body tissue and irradiate the body tissue with excitation light corresponding to the fluorescence wavelength of the reagent to obtain a fluorescent image, for example. The light source device 5043 can be cable of supplying narrow band light and/or excitation light for such special light observation.

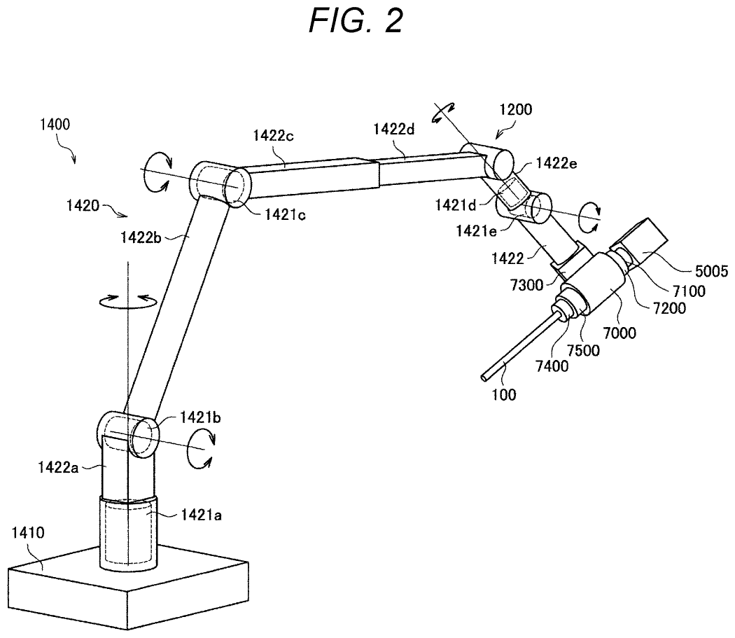

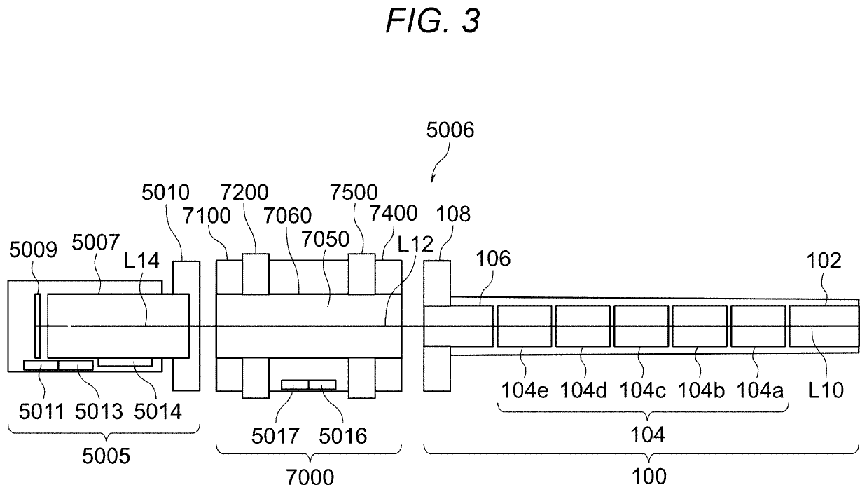

Details of a support arm device 1400 including a robot arm 1420 and an arm control device 1350 and the endoscope unit 5006 will be described with reference to FIGS. 2 and 3. FIG. 2 is a schematic diagram illustrating a configuration in which the holding unit portion 7000 that independently controls the rotation of an oblique-viewing endoscope 100 and the rotation of a camera head 7100 is provided at the distal end of the robot arm 1420. FIG. 3 is a diagram illustrating a configuration example of the endoscope unit 5006. Note that the holding unit portion 7000 according to the present embodiment corresponds to an endoscope holding device.

### (Arm and Arm Control Device)

As illustrated in FIG. 2, the support arm device 1400 according to the present embodiment includes a base 1410 and the robot arm 1420. The base 1410 is a base of the support arm device 1400, and the robot arm 1420 is extended from the base 1410. Then, the holding unit portion 7000 is formed at an end portion of robot arm 1420.

Furthermore, although not illustrated in FIG. 2, a control unit that integrally controls the support arm device 1400 may be provided in the base 1410, and driving of the robot arm 1420 may be controlled by the control unit. The control unit includes, for example, various signal processing circuits such as a CPU and a DSP.

The robot arm 1420 includes a plurality of active joints 1421a to 1421e, a plurality of links 1422a to 1422e, and the endoscope unit 5006 as a distal end unit provided at the distal end of the robot arm 1420.

### (Endoscope Unit)

As illustrated in FIG. 3, in the endoscope unit 5006 according to the present embodiment, the holding unit portion 7000 can be attached between the oblique-viewing endoscope 100 which is a rigid endoscope and the camera head 5005. That is, the endoscope unit 5006 includes the oblique-viewing endoscope 100, the camera head 5005, and the holding unit portion 7000. In addition, the holding unit portion 7000 rotatably holds the oblique-viewing endoscope 100 and the camera head 5005. Furthermore, a commercially available general-purpose product can be used for each of the camera head 5005 and the oblique-viewing endoscope 100 according to the present embodiment. Therefore, the camera head 5005 and the oblique-viewing endoscope 100 according to the present embodiment are connectable without the holding unit portion 7000. As described above, the oblique-viewing endoscope 100 according to the present embodiment corresponds to the endoscope. Furthermore, the camera head 5005 according to the present embodiment corresponds to an imaging device.

Note that in the present embodiment, an example in which the oblique-viewing endoscope 100, the camera head 5005, and the holding unit portion 7000 are detachable will be described, but the present invention is not limited thereto. For example, all of these optical components may be fixed at all times. Alternatively, at least one of the oblique-viewing endoscope 100 and the camera head 5005 may be fixed to the holding unit portion 7000.

### (Rigid Endoscope)

The oblique-viewing endoscope 100 is, for example, a rigid endoscope, and includes an objective lens 102, a first relay lens system 104, an eyepiece 106, and an attachment portion 108. Note that the oblique-viewing endoscope 100 is capable of observing an object in the body cavity or the like with the naked eye through the eyepiece 106. An optical axis L10 is an optical axis of the objective lens 102 and the first relay lens system 104. That is, the optical axis L10 according to the present embodiment corresponds to a first optical axis. Note that the oblique-viewing endoscope 100 according to the present embodiment is an oblique-viewing endoscope, but is not limited thereto. For example, a forward-viewing endoscope may be used.

The objective lens 102 includes, for example, a front lens group 2 and a rear lens group so that a wide range can be observed. The objective lens 102 forms a real image obtained by reducing an object in the body cavity or the like.

Moreover, the first relay lens system 104 includes a plurality of relay lens systems 104a to 104e. Each of the plurality of relay lens systems 104a to 104e is formed of, for example, a lens group. As a result, an imaging system having a long distance is implemented by connecting the plurality of relay lens systems 104a to 104e so that the state of the object in the body cavity or the like can be observed outside the body cavity.

As described above, each of the relay lens systems 104a to 104e is designed so that the light beam states on the incident side and the emission side are the same, and the relay lens systems 104a to 104e repeatedly form the real image formed by the objective lens 102 to transmit the real image.

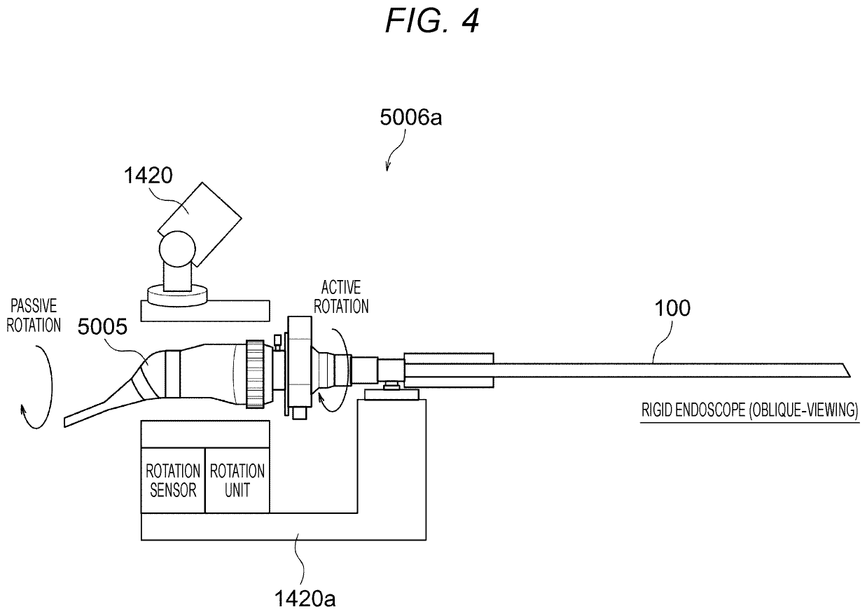

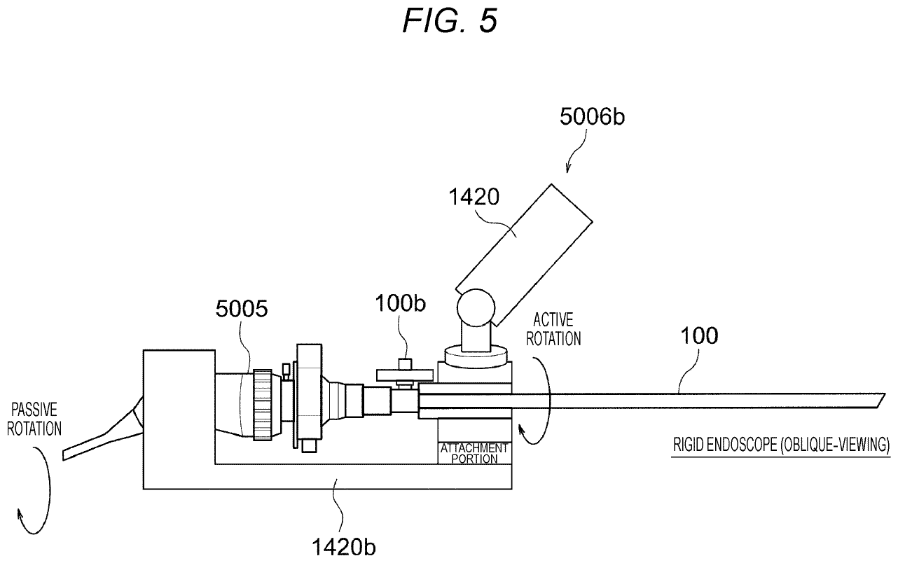

For example, the eyepiece 106 virtually forms a real image transmitted from first relay lens system 104, and magnifies and forms the real image rearward from the eyepiece 106 toward the holding unit portion 7000. The attachment portion 108 is a coupling portion that is detachably coupled to a second coupling portion 7400 of the holding unit portion 7000. Here, a problem in the case of not including the holding unit portion 7000 will be described with reference to FIGS. 4 and 5.

FIG. 4 is a schematic external view illustrating an endoscope unit 5006a of a first comparative example. FIG. 4 illustrates an example in which a camera head 5005 is held by a holding portion 1420a of a robot arm 1420. As illustrated in FIG. 4, when the camera head 5005 is to be held by the holding portion 1420a, the distal end portion becomes large due to the large camera head 5005. Therefore, when the camera head 5005 and the oblique-viewing endoscope 100 are to be held by the robot arm 1420 in a rotatable manner, there is a possibility that the camera head 5005 interferes with the arm of the operator and hinders the surgery.

FIG. 5 is a schematic external view illustrating an endoscope unit 5006b of a second comparative example. FIG. 5 illustrates an example in which a holding portion 1420b of a robot arm 1420 holds the side of an oblique-viewing endoscope 100. As illustrated in FIG. 5, the oblique-viewing endoscope 100 is a portion having a small diameter of the endoscope, but when the lens barrel portion of the oblique-viewing endoscope 100 is to be held by the holding portion 1420b, it is necessary to hold the oblique-viewing endoscope 100 while avoiding a light guide portion 100b into which light from a light source device 5043 (see FIG. 1) is introduced. Therefore, the lens barrel portion protruding from the distal end portion of the holding portion 1420b is shortened. That is, when the protruding amount of the lens barrel from a holding unit portion 7000b is small, the amount that can be inserted into the body of the patient decreases, and there is a possibility that the field of view desired by the operator cannot be obtained.

### (Holding Unit Portion)

In order to solve the above-described problems illustrated in FIGS. 4 and 5, the first embodiment provides the holding unit portion 7000 that curbs interference with the arm of the operator and curbs reduction in the protruding amount of the lens barrel when the robot arm 1420 rotatably holds each of the oblique-viewing endoscope 100 and the camera head 5005. That is, the holding unit portion 7000 has a relay optical system and is provided between the oblique-viewing endoscope 100 and the camera head 5005.

More specifically, as illustrated in FIG. 3, the holding unit portion 7000 includes a perspective control unit 5016, a camera head rotation control unit 5017, a second relay optical system 7050, a housing 7060, a camera head attachment portion 7100, a camera head rotation drive unit 7200, an oblique-viewing endoscope attachment portion 7400, and an oblique-viewing endoscope rotation drive unit 7500.

The perspective control unit 5016 controls the oblique-viewing endoscope rotation drive unit 7500 to rotate the oblique-viewing endoscope 100. The camera head rotation control unit 5017 controls the camera head rotation drive unit 7200 to rotate the camera head 5005. Note that details of the perspective control unit 5016 and the camera head rotation control unit 5017 will be described later.

The second relay optical system 7050 is provided in the housing 7060 and moves the exit pupil of the oblique-viewing endoscope 100 to the position of the entrance port of the camera head 5005. That is, the housing 7060 accommodates the second relay optical system 7050 therein and is disposed at an end portion of the robot arm 1420. As described above, the camera head 5005 and the oblique-viewing endoscope 100 according to the present embodiment are connectable without the holding unit portion 7000. Therefore, the second relay optical system 7050 has a configuration according to an afocal optical system, and relays the exit pupil of the oblique-viewing endoscope 100 to the camera head 5005 at equal magnification, for example. As a result, the image captured by the camera head 5005 is equivalent in size in a case where the image is captured via the holding unit portion 7000 and in a case where the image is captured without the holding unit portion 7000.

The camera head attachment portion 7100 detachably attaches the camera head 5005 to the camera head rotation drive unit 7200 via an attachment portion 5010 of the camera head 5005. The camera head attachment portion 7100 is fixed so that the optical axis L10 of the oblique-viewing endoscope 100 coincides with an optical axis L12 of an optical system 5007 of the camera head 5005.

The camera head rotation drive unit 7200 includes an actuator such as a motor, and rotates the camera head 5005 with respect to the main body of the holding unit portion 7000. That is, the camera head rotation drive unit 7200 rotates the camera head 5005 with respect to the optical axis L12 of the second relay optical system 7050, the camera head 5005 being fixed such that light emitted from the second relay optical system 7050 enters the camera head 5005. Note that the camera head attachment portion 7100 according to the present embodiment corresponds to a second attachment portion, and the camera head rotation drive unit 7200 corresponds to a second rotation unit.

In addition, the attachment portion 5010 can be coupled to the attachment portion 108. As a result, as described above, the camera head 5005 and the oblique-viewing endoscope 100 can be directly connected without the holding unit portion 7000. Therefore, it is also possible to accommodate surgical methods that do not use the robot arm 1420.

The oblique-viewing endoscope attachment portion 7400 detachably attaches the oblique-viewing endoscope 100 to the oblique-viewing endoscope rotation drive unit 7500 via the attachment portion 108. The oblique-viewing endoscope attachment portion 7400 is fixed such that the optical axis L10 of the oblique-viewing endoscope 100 coincides with the optical axis L12 of the second relay optical system 7050. That is, the oblique-viewing endoscope attachment portion 7400 is fixed such that light emitted from the oblique-viewing endoscope 100 enters the second relay optical system 7050. Note that the oblique-viewing endoscope attachment portion 7400 according to the present embodiment corresponds to a first attachment portion, and the oblique-viewing endoscope rotation drive unit 7500 corresponds to a first rotation unit.

The oblique-viewing endoscope rotation drive unit 7500 includes an actuator such as a motor, and rotates the oblique-viewing endoscope 100 with respect to the main body of the holding unit portion 7000. That is, the oblique-viewing endoscope rotation drive unit 7500 rotates the oblique-viewing endoscope 100 with respect to the optical axis L12 of the second relay optical system 7050, the oblique-viewing endoscope 100 being fixed so that light emitted therefrom enters the second relay optical system 7050.

Furthermore, by arranging the configuration of the housing 7060, the camera head rotation drive unit 7200, the oblique-viewing endoscope rotation drive unit 7500, and the like in a sealed structure having high airtightness and waterproofness, the holding unit portion 7000 can have resistance to autoclave sterilization processing.

### (Camera Head)

The camera head 5005 includes a lens unit 5007, an imaging unit 5009, a drive unit 5011, a communication unit 5013, and an endoscope unit control unit 5014. The lens unit 5007 is an optical system provided at a connection portion with the holding unit portion 7000. Observation light taken in from the distal end of the oblique-viewing endoscope 100 is guided to the camera head 5005 via the second relay optical system 7050 of the holding unit portion 7000, and is incident on the lens unit 5007.

The lens unit 5007 includes a combination of a plurality of lenses including a zoom lens and a focus lens. The lens unit 5007 includes an autofocus (AF) optical system. Furthermore, positions on the optical axes of the zoom lens and the focus lens are movable to adjust the magnification and focus of a captured image.

The imaging unit 5009 includes the imaging element, and is disposed at the subsequent stage of the lens unit 5007. The observation light that passes through the lens unit 5007 is condensed on the light-receiving surface of the imaging element, and an image signal corresponding to an observation image is generated by photoelectric conversion. The image signal generated by the imaging unit 5009 is provided to the communication unit 5013.

As the imaging element forming the imaging unit 5009, for example, a complementary metal oxide semiconductor (CMOS) type image sensor, which has a Bayer array and is capable of color imaging, is used. Note that an element capable of capturing a high-resolution image of 4K or higher may be used as the imaging element, for example. The image of the surgical site at high resolution is obtained, whereby the operator 5067 can grasp a state of the surgical site in further detail, and can proceed with the surgery more smoothly.

The drive unit 5011 controls the position of each lens of the lens unit 5007 according to the endoscope unit control unit 5014. The endoscope unit control unit 5014 controls the entire endoscope unit 5006 in cooperation with the CCU 5039 (see FIG. 1). Details of the endoscope unit control unit 5014 will be described later. Note that the camera head 5005 can be made to have resistance to autoclave sterilization processing by arranging configurations of the lens unit 5007, the imaging unit 5009, and the like in a sealed structure having high airtightness and waterproofness.

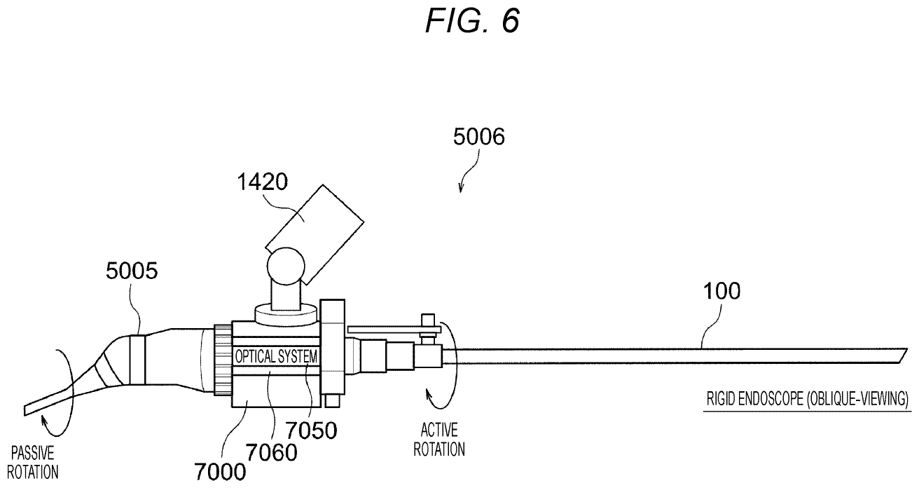

FIG. 6 is a schematic external view illustrating the endoscope unit 5006 according to the present embodiment. As illustrated in FIG. 6, in the endoscope unit 5006 according to the present embodiment, the second relay optical system 7050 is disposed between the camera head 5005 and the oblique-viewing endoscope 100. As a result, it is not necessary to directly fit the camera head 5005 and the oblique-viewing endoscope 100. Furthermore, the housing 7060 having the second relay optical system 7050 therein can have a smaller structure than the structure of the camera head 5005. As a result, the holding unit portion 7000 provided in the robot arm 1420 can be downsized, and the rotation mechanism can be accommodated in the holding unit portion 7000. As can be seen from the above, while the robot arm 1420 has the rotation mechanisms of the camera head 5005 and the oblique-viewing endoscope 100 in the holding unit portion 7000 at the distal end, the holding portion can be downsized.

### (Functional Component)

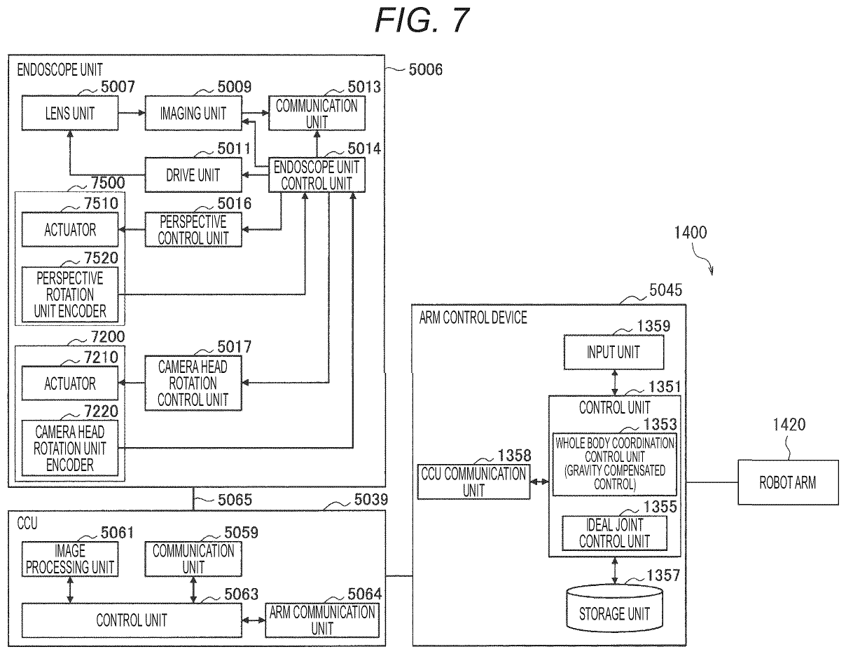

FIG. 7 is a block diagram illustrating an example of functional components of the endoscope unit 5006, the CCU 5039, and the arm control device 5045 illustrated in FIG. 1. Functions of the endoscope unit 5006, the CCU 5039, and the arm control device 5045 in the configuration example illustrated in FIG. 2 will be described in more detail with reference to FIG. 7.

As described above, the endoscope unit 5006 includes the camera head 5005, the holding unit portion 7000, and the oblique-viewing endoscope 100. That is, the endoscope unit 5006 includes, as functional components, the lens unit 5007, the imaging unit 5009, the drive unit 5011, the communication unit 5013, the endoscope unit control unit 5014, the perspective control unit 5016, the camera head rotation control unit 5017, the camera head rotation drive unit 7200, and the oblique-viewing endoscope rotation drive unit 7500.

Furthermore, the CCU 5039 includes, as functional components thereof, a communication unit 5059, an image processing unit 5061, a control unit 5063, and an arm communication unit 5064. The camera head 5005 and the CCU 5039 are communicably connected to each other in both directions by a transmission cable 5065.

Furthermore, the arm control device 5045 is a control device of the support arm device 1400, and includes a control unit 1351, a storage unit 1357, and an input unit 1359. Furthermore, the control unit 1351 includes a whole body coordination control unit 1353 and an ideal joint control unit 1355.

### (Camera Head of Endoscope Unit)

First, functional components of the camera head 5005 of the endoscope unit 5006 will be described. The communication unit 5013 receives a control signal for controlling driving of the camera head 5005 from the CCU 5039. The control signal includes, for example, information regarding imaging conditions such as information specifying a frame rate of the captured image, information specifying an exposure value at the time of imaging, and/or information specifying the magnification and focus of the captured image. The communication unit 5013 provides the received control signal to the endoscope unit control unit 5014.

The endoscope unit control unit 5014 controls the drive unit 5011 according to the control signal of the CCU 5039 such that the lens unit 5007 condenses the observation light on the light receiving surface of the imaging element of the imaging unit 5009. That is, the drive unit 5011 includes an actuator, and moves the zoom lens and the focus lens of the lens unit 5007 by a predetermined distance along an optical axis under the control of the endoscope unit control unit 5014. As a result, the magnification and the focus of the image captured by the imaging unit 5009 can be appropriately adjusted.

Furthermore, the endoscope unit control unit 5014 supplies the image signal generated by the imaging unit 5009 to the CCU 5039 via the communication unit 5013. That is, the communication unit 5013 includes a communication device for transmitting and receiving various kinds of information to and from the CCU 5039. The communication unit 5013 transmits the image signal obtained from the imaging unit 5009 as RAW data to the CCU 5039 via the transmission cable 5065.

The imaging conditions such as the frame rate, the exposure value, the magnification, and the focus of the imaging unit 5009 are automatically set by the control unit 5063 of the CCU 5039 on the basis of the acquired image signal. That is, a so-called auto exposure (AE) function, auto-focus (AF) function, and auto white balance (AWB) function are installed in the endoscope unit 5006 and are executed via the endoscope unit control unit 5014.

### (Holding Unit Portion of Endoscope Unit)

Next, functional components of the holding unit portion 7000 of the endoscope unit 5006 will be described. The perspective control unit 5016 of the holding unit portion 7000 drives an actuator 7510 on the basis of a command from the endoscope unit control unit 5014. The actuator 7510 and a perspective rotation unit encoder 7520 are provided in the oblique-viewing endoscope rotation drive unit 7500. The endoscope unit control unit 5014 drives the actuator 7510 on the basis of a rotation angle of the actuator 7510 detected by the perspective rotation unit encoder 7520, and controls the rotation of the oblique-viewing endoscope 100 around the optical axis L12. Note that the actuator 7510 according to the present embodiment corresponds to a first electric motor unit.

Furthermore, the camera head control unit 5017 drives an actuator 7210 on the basis of a command from the endoscope unit control unit 5014. The actuator 7210 and a camera head rotation unit encoder 7220 are provided in the camera head rotation drive unit 7200. The endoscope unit control unit 5014 controls the rotation of the camera head 5005 around the optical axis L14 on the basis of the rotation angle of the actuator 7210 detected by the camera head rotation unit encoder 7220. Note that the actuator 7210 according to the present embodiment corresponds to a second electric motor unit.

With the above configuration, rotation of the oblique-viewing endoscope 100 and the camera head 5005 can be independently controlled with respect to the main body of the holding unit portion 7000.

### (CCU)

Next, functional components of the CCU 5039 will be described. The communication unit 5059 includes a communication device for transmitting and receiving various types of information to and from the camera head 5005. The communication unit 5059 receives the image signal transmitted from the camera head 5005 via the transmission cable 5065. The communication unit 5059 provides the image signal converted into an electric signal to the image processing unit 5061. Furthermore, the communication unit 5059 transmits a control signal for controlling driving of the camera head 5005 to the camera head 5005. Similarly, the communication unit 5059 transmits a control signal for controlling rotation of the camera head 5005 and the oblique-viewing endoscope 100 to the holding unit portion 7000 via the endoscope unit control unit 5014.

The image processing unit 5061 performs various types of image processing on the image signal that is RAW data transmitted from the camera head 5005. The image processing includes various types of known signal processing such as, for example, development processing, high image quality processing (band emphasizing processing, super resolution processing, noise reduction (NR) processing, and/or camera shake correction processing), enlargement processing (electronic zoom processing), and/or the like. Furthermore, the image processing unit 5061 performs detection processing on the image signal for performing AE, AF, and AWB.

The image processing unit 5061 includes a processor such as a CPU or GPU, and the image processing and detection processing described above can be performed by the processor operating in accordance with a predetermined program. Note that in a case where the image processing unit 5061 includes a plurality of GPUs, the image processing unit 5061 appropriately divides information related to the image signal and performs the image processing in parallel by the plurality of GPUs.

The control unit 5063 performs various controls regarding imaging of the surgical site by the endoscope unit 5006 and display of the captured image. For example, the control unit 5063 generates a control signal for controlling driving of the camera head 5005. At this time, in a case where the imaging conditions are is input by the user, the control unit 5063 generates the control signal on the basis of the input by the user. Alternatively, in a case where the AE function, the AF function, and the AWB function are installed in the endoscope unit 5006, the control unit 5063 generates a control signal by appropriately calculating the optimum exposure value, focal length, and white balance depending on a result of the detection processing by the image processing unit 5061.

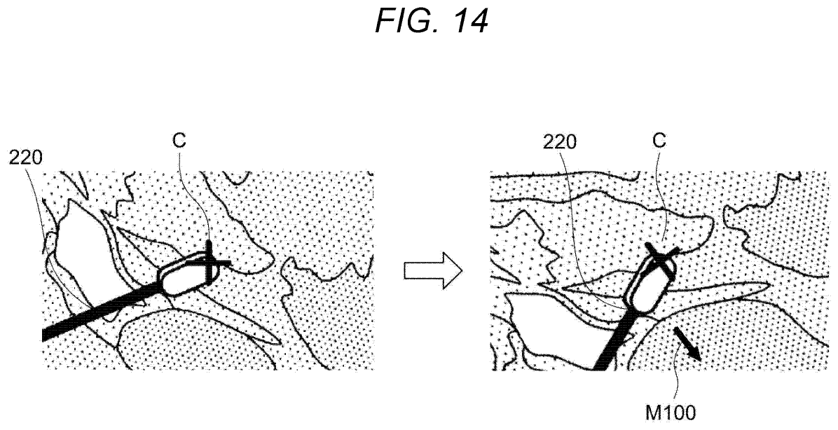

Furthermore, the control unit 5063 causes the display device 5041 to display the image of the surgical site on the basis of the image signal subjected to the image processing by the image processing unit 5061. At this time, the control unit 5063 recognizes various objects in the surgical site image by using various image recognition technologies. For example, the control unit 5063 detects a shape, a color, and the like of an edge of an object included in the surgical site image, thereby being able to recognize a surgical tool such as forceps, a specific living-body site, bleeding, mist at the time of use of the energy treatment tool 5021, and the like. Furthermore, the image processing unit 5061 can superimpose marks M100 and M102 (see FIGS. 14 and 16) and the like described later on an image.

When causing the display device 5041 to display the image of the surgical site, the control unit 5063 causes various types of surgery assistance information to be superimposed and displayed on the image of the surgical site by using a result of the recognition. The surgery assistance information is superimposed and displayed, and presented to the operator 5067, whereby the surgery can be performed more safely and reliably. The arm communication unit 5064 communicates with the arm control device 5045. Note that details of rotation control of the CCU 5039 will be described later.

### (Arm Control Device)

The control unit 1351 of the arm control device 5045 includes, for example, various signal processing circuits such as a CPU and a DSP. The control unit 1351 integrally controls the arm control device 5045, and performs various calculations for controlling driving of the robot arm 1420 in the support arm device 1400. Specifically, the control unit 1351 includes the whole body coordination control unit 1353 and the ideal joint control unit 1355. The whole body coordination control unit 1353 performs various calculations in whole body coordination control in order to control driving of the actuators 1430 provided in the active joints 1421a to 1421f of the robot arm 1420 of the support arm device 1400. The ideal joint control unit 1355 performs various calculations in ideal joint control that achieves an ideal response to the whole body coordination control by correcting the influence of disturbance. The storage unit 1357 may be, for example, a storage element such as a random access memory (RAM) or a read only memory (ROM), or may be a semiconductor memory, a hard disk, or an external storage device.

The input unit 359 is an input interface for the user to input information, commands, and the like regarding the driving control of the support arm device 400 to the control unit 351. The input unit 359 includes, for example, operation means operated by the user such as a lever and a pedal, and the position, speed, and the like of each component of the robot arm 1420 may be set as the instantaneous motion purpose according to the operation of the lever, the pedal, and the like. Such an input unit 359 may include, for example, an operation means operated by the user such as a mouse, a keyboard, a touch panel, a button, and a switch in addition to a lever and a pedal. Furthermore, the arm control device 5045 and the CCU 5039 can transmit and receive information to and from each other by communication between a CCU communication unit 1358 and the arm communication unit 5064.

### (Rotation Control of CCU)

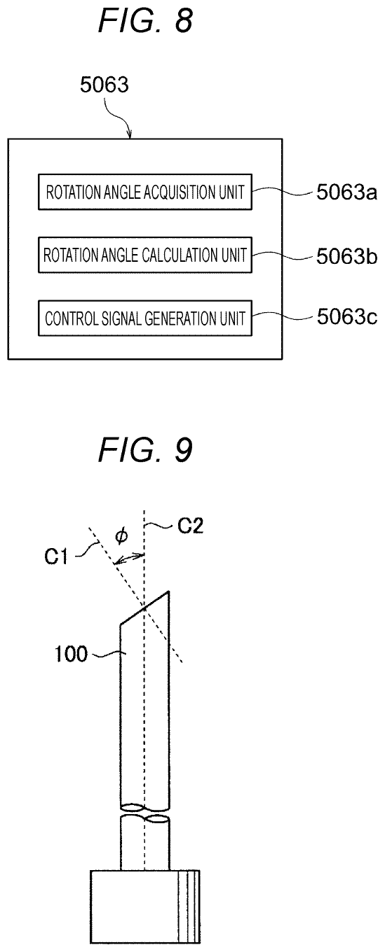

Here, details of rotation control of the CCU 5039 will be described. FIG. 8 is a block diagram of a configuration example related to rotation control with respect to the holding unit portion 7000 in the control unit 5063. As illustrated in FIG. 8, the control unit 5063 includes a rotation angle acquisition unit 5063a, a rotation angle calculation unit 5063b, and a control signal generation unit 5063c.

The rotation angle acquisition unit 5063a acquires the rotation angle of the actuator 7510 detected by the perspective rotation unit encoder 7520 of the holding unit portion 7000. When the oblique-viewing endoscope 100 is rotated to visually recognize a desired observation target, the rotation angle calculation unit 5063b calculates a rotation angle for appropriately controlling the top and bottom of the image by rotating the camera head 5005. Note that the rotation angle acquisition unit 5063a according to the present embodiment corresponds to a first rotation angle acquisition unit.

The control signal generation unit 5063c supplies a control signal based on the rotation angle calculated by the rotation angle calculation unit 5063b to the camera head control unit 5017 via the communication unit 5059 and the endoscope unit control unit 5014. The camera head control unit 5017 drives the actuator 7210 on the basis of a command from the endoscope unit control unit 5014. That is, the control signal generation unit 5063c transmits a control signal for controlling rotation of the camera head 5005 to the camera head control unit 5017 via the communication unit 5059 and the endoscope unit control unit 5014. In this manner, the actuator 7210 rotates the camera head 5005 around the optical axis L14 on the basis of the information of the rotation angle of the oblique-viewing endoscope 100 acquired by the rotation angle acquisition unit 5063a.

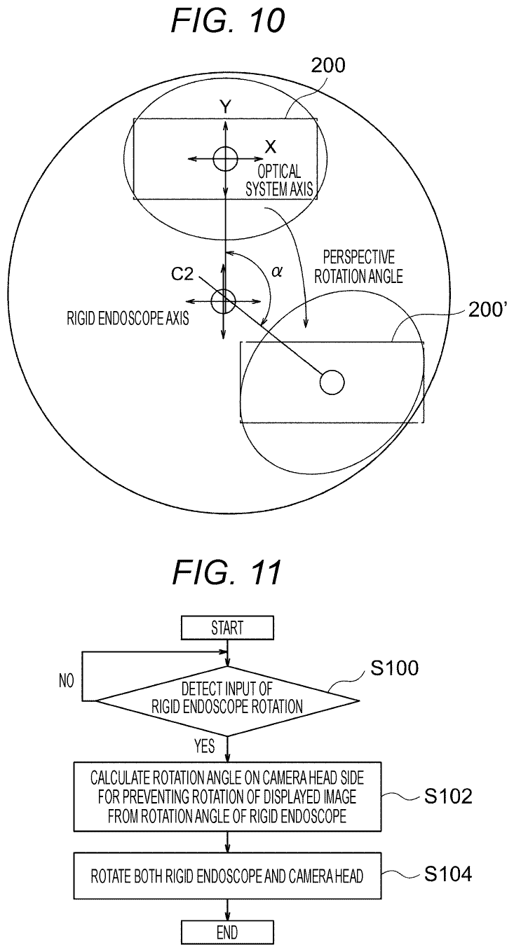

A control example of the rotation angle calculation unit 5063b will be described in more detail with reference to FIGS. 9 and 10. FIG. 9 is a schematic diagram illustrating an appearance of the oblique-viewing endoscope 100. FIG. 10 is a schematic diagram illustrating how a visual field 200 displayed on the display device 5041 changes due to the perspective rotation.

As illustrated in FIG. 9, in the oblique-viewing endoscope 100, a direction (C1) of the objective lens toward the subject has a predetermined angle φ with respect to the longitudinal direction (scope axis C2) of the oblique-viewing endoscope 100. That is, in the oblique-viewing endoscope 100, the objective optical system forms an angle with respect to the eyepiece optical system of the scope. In the oblique-viewing endoscope 100, an operation of rotating (hereinafter referred to as perspective rotation) the oblique-viewing endoscope 100 with the scope axis C2 as a rotation axis is performed for observation. By performing the perspective rotation, it is possible to obtain a wraparound visual field and vertical and horizontal peripheral visual fields. Note that the scope axis C2 corresponds to the optical axis L10.

As illustrated in FIG. 10, the visual field 200 of the oblique-viewing endoscope 100 is displayed on the monitor of the display device 5041 (see FIG. 1) before the oblique-viewing endoscope 100 rotates. A visual field 200′ of the oblique-viewing endoscope 100 indicates a state of being displayed on the monitor of the display device 5041 (see FIG. 1) after perspective rotation of the oblique-viewing endoscope by a perspective rotation angle α. In this way, the vertical directions of the field of visual field 200 and the field of visual field 200′ are controlled to coincide.

More specifically, the rotation angle acquisition unit 5063a acquires the perspective rotation angle α of the actuator 7510 detected by the perspective rotation unit encoder 7520 (see FIG. 7). The rotation angle calculation unit 5063b calculates a rotation angle γ of the camera head 5005 for maintaining the angle of the camera head 5005 in the vertical direction on the basis of the perspective rotation angle α. The control signal generation unit 5063c supplies a control signal based on the rotation angle γ calculated by the rotation angle calculation unit 5063b to the camera head control unit 5017. That is, the actuator 7510 rotates the camera head 5005 with respect to the optical axis L14 such that the camera head 5005 maintains a predetermined rotation angle, that is, an angle in the vertical direction with respect to the optical axis L14 on the basis of the information of the perspective rotation angle α of the oblique-viewing endoscope 100. As described above, by controlling the two axes of the perspective rotation a and the rotation angle γ of the camera head 5005, the CCU 5039 can obtain vertical and horizontal peripheral visual fields while maintaining the vertical direction of the visual field 200.

FIG. 11 is a flowchart illustrating an example of rotation control of the oblique-viewing endoscope 100 and the camera head 5005. As illustrated in FIG. 11, the rotation angle acquisition unit 5063a sequentially acquires the perspective rotation angle of the actuator 7510 detected by the perspective rotation unit encoder 7520 (see FIG. 7), and the rotation angle calculation unit 5063b determines whether or not the oblique-viewing endoscope 100 has rotated on the basis of the perspective rotation angle acquired by the rotation angle acquisition unit 5063a (step S100). If the rotation angle calculation unit 5063b determines that the rotation angle of the oblique-viewing endoscope 100 has not changed (NO in step S100), the rotation angle calculation unit 5063b repeats the processing of step S100.

On the other hand, if the rotation angle calculation unit 5063b determines that the rotation angle of the oblique-viewing endoscope 100 has changed (YES in step S100), the rotation angle calculation unit 5063b calculates the rotation angle γ of the camera head 5005 for maintaining the angle in the vertical direction of the camera head 5005 on the basis of the acquired perspective rotation angle α (step S102).

Next, the control signal generation unit 5063c of the CCU 5039 transmits a control signal related to the rotation angle γ of the camera head 5005 to the endoscope unit control unit 5014 of the endoscope unit 5006. Then, the endoscope unit control unit 5014 controls driving of the actuator 7510 via the perspective control unit 5016 (step S104).

In this manner, according to the input from the input unit 359, the oblique-viewing endoscope 100 is rotated to visually recognize a desired observation target, and the camera head 5005 is rotated to appropriately control the top and bottom of the image.

As described above, in the endoscope unit 5006 according to the present embodiment, the holding unit portion 7000 having the relay optical system 7050 is disposed between the camera head 5005 and the oblique-viewing endoscope 100. Since the housing 7060 having the relay optical system 7050 therein can reduce the diameter of the housing 7060 with respect to the structure of the camera head 5005, the holding unit portion 7000 provided in the robot arm 1420 can be downsized, and the rotation mechanism portion can be accommodated in the holding unit portion 7000. As a result, the robot arm 1420 can be downsized while having the rotation mechanisms of the camera head 5005 and the oblique-viewing endoscope 100 in the holding unit portion 7000 at the distal end. In this manner, by providing the holding unit portion 7000, the endoscope unit 5006 is configured to curb interference with the arm of the operator and not to hinder the surgery.

### Second Embodiment

An endoscope unit 5006c according to a second embodiment is different from the endoscope unit 5006 according to the first embodiment in that a camera head 5005 is manually rotatable by the operator in addition to the automatic control according to the first embodiment. Hereinafter, differences from the endoscope unit 5006 according to the first embodiment will be described.

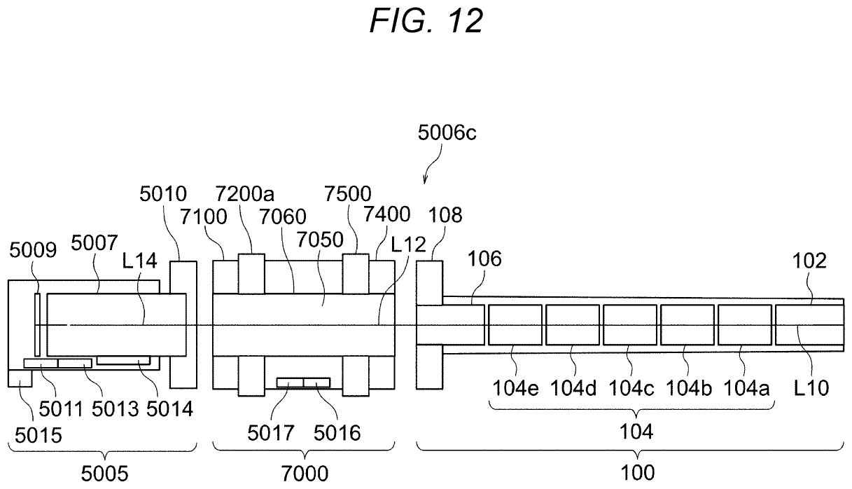

FIG. 12 is a diagram illustrating a configuration example of the endoscope unit 5006a according to the second embodiment. As illustrated in FIG. 12, the camera head 5005 further includes an acceleration sensor 5015. The acceleration sensor 5015 can detect, for example, an angle in a gravity direction with respect to a reference line or acceleration in a tangential direction of a circle on a plane orthogonal to an optical axis with the optical axis of the camera head 5005 as a center point.

An actuator 7210 (see FIG. 7) of a camera head rotation drive unit 7200a is also rotatable in response to an external force applied to the camera head 5005, for example. As a result, the operator can rotate the camera head 5005 with respect to a holding unit portion 7000.

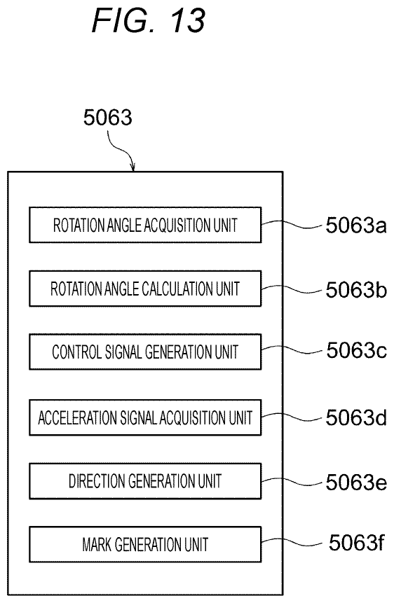

With reference to FIGS. 13 and 14, rotation control of the holding unit portion 7000 in a control unit 5063 according to the second embodiment will be described. FIG. 13 is a block diagram of a configuration example related to rotation control of the holding unit portion 7000 in the control unit 5063 according to the second embodiment. As illustrated in FIG. 13, the control unit 5063 according to the second embodiment further includes an acceleration signal acquisition unit 5063d, a direction generation unit 5063e, and a mark generation unit 5063f.

FIG. 14 is a diagram for describing an outline of the present embodiment. FIG. 14 illustrates an image (hereinafter also referred to as an endoscopic image) obtained by the endoscope unit 5006a illustrated in FIG. 13. In FIG. 13, forceps 220 and an image center C are displayed. The left figure is an image obtained in the current position and posture, and the right figure is an image obtained after movement by endoscope control processing according to the present embodiment. That is, in the right diagram, the oblique-viewing endoscope 100 rotates. A mark M100 according to the present embodiment is a mark indicating a vertically downward direction.

As illustrated in FIG. 13, the acceleration signal acquisition unit 5063 acquires an output signal of the acceleration sensor 5015. The direction generation unit 5063e generates the rotation angle of the camera head 5005 from the gravity direction using the output signal of the acceleration sensor 5015. Then, the direction generation unit 5063e outputs an output signal having information of the rotation angle from the gravity direction to the mark generation unit 5063f. Note that the gravity direction of the camera head 5005 according to the present embodiment is a direction in which the top and bottom of a captured image of the camera head 5005 when displayed is in a predetermined orientation, such as the upward direction of gravity, when the camera head 5005 is turned in the gravity direction.

The mark generation unit 5063f generates a mark M100 indicating the gravity direction with respect to the captured image of the camera head 5005 on the basis of the output signal of the direction generation unit 5063e. Then, the mark generation unit 5063f outputs a signal including information of the mark M100 to an image processing unit 5061 (see FIG. 7). As a result, the image processing unit 5061 generates an image in which the mark M100 indicating the gravity direction is superimposed on the captured image of the camera head 5005. A monitor of a display device 5041 displays the captured image on which the mark M100 indicating the gravity direction is superimposed.

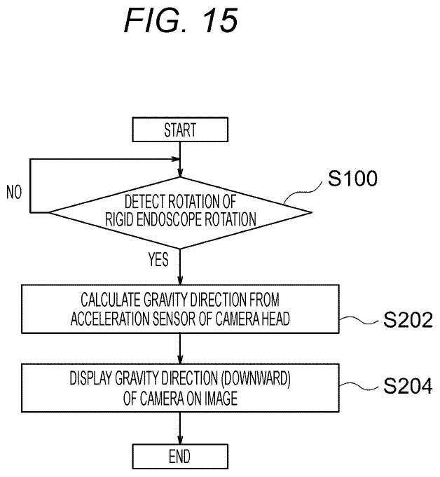

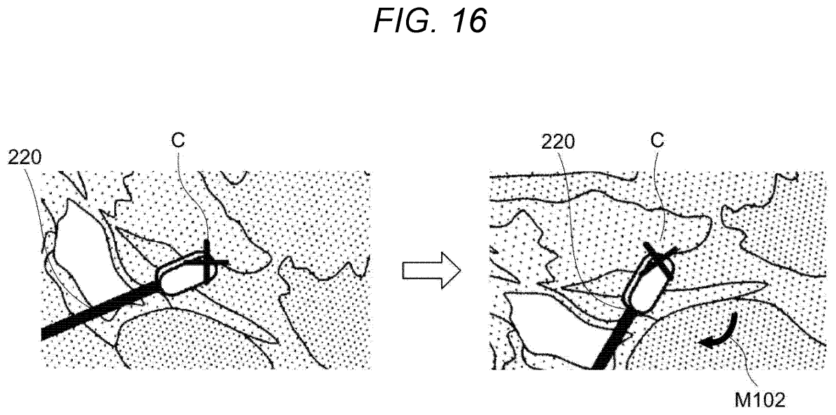

FIG. 15 is a flowchart illustrating a control example according to the second embodiment. As illustrated in FIG. 15, the rotation angle calculation unit 5063b determines whether or not the oblique-viewing endoscope 100 has rotated (step S100). If the rotation angle calculation unit 5063b determines that the rotation angle of the oblique-viewing endoscope 100 has not changed (NO in step S100), the rotation angle calculation unit 5063b repeats the processing of step S100.

On the other hand, if the rotation angle calculation unit 5063b determines that the rotation angle of the oblique-viewing endoscope 100 has changed (YES in step S100), the direction generation unit 5063e outputs an output signal having information of the rotation angle from the gravity direction to the mark generation unit 5063f on the basis of the acquired output signal of the acceleration sensor 5015 (step S202).

Next, the mark generation unit 5063f generates the mark M100 using the information of the rotation angle from the gravity direction, and outputs the mark M100 to the image processing unit 5061. As a result, the image processing unit 5061 generates an image in which the mark M100 indicating the gravity direction is superimposed on the captured image of the camera head 5005, and the display device 5041 displays the image on which the mark M100 indicating the gravity direction is superimposed (step S204).

As described above, in the endoscope unit 5006 according to the present embodiment, the camera head 5005 is manually rotatable and controllable by the operator. In this case, the direction generation unit 5063e outputs an output signal having information of the rotation angle from the gravity direction to the mark generation unit 5063f, the mark generation unit 5063f outputs an output signal having information of the mark M100 indicating the gravity direction to the image processing unit 5061, and the image processing unit 5061 generates an image in which the mark M100 indicating the gravity direction is superimposed on the captured image of the camera head 5005. As a result, the operator can rotate the camera head 5005 with respect to the holding unit portion 7000 according to the direction indicated by the mark M100. Therefore, the operator can rotate the oblique-viewing endoscope 100 to visually recognize a desired observation target, and rotate the camera head 5005 to appropriately control the top and bottom of the image.

### Modification 1 of Second Embodiment

An endoscope unit 5006c according to Modification 1 of the second embodiment is different from the endoscope unit 5006 according to the second embodiment in that a direction in which a camera head 5005 is rotated can be further indicated by a mark. Hereinafter, differences from the endoscope unit 5006 according to the second embodiment will be described.

FIG. 16 is a diagram for describing an outline of Modification 1 of the second embodiment. FIG. 16 illustrates an image (hereinafter also referred to as an endoscopic image) obtained by the endoscope unit 5006a illustrated in FIG. 12. In FIG. 16, forceps 220 and an image center C are displayed. The left figure is an image obtained in the current position and posture, and the right figure is an image obtained after movement by endoscope control processing according to the present embodiment. That is, in the right diagram, the oblique-viewing endoscope 100 rotates. A mark M102 indicates a direction in which the camera head 5005 is rotated to direct the top and bottom of the image in an appropriate direction. Additionally, the length of the mark M102 corresponds to the rotation angle.

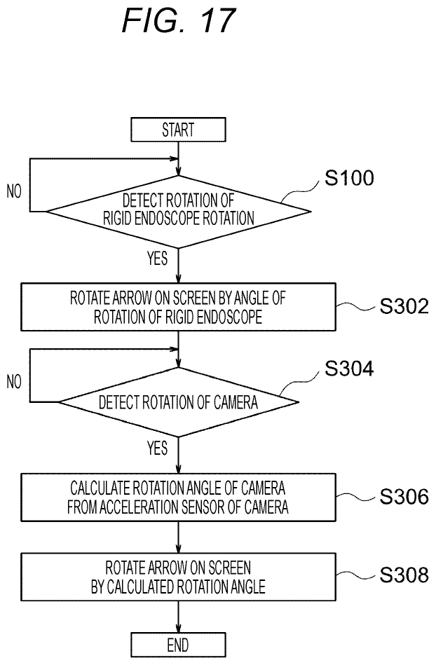

FIG. 17 is a flowchart illustrating a control example according to Modification 1 of the second embodiment. As illustrated in FIG. 17, a rotation angle calculation unit 5063b determines whether or not the oblique-viewing endoscope 100 has rotated (step S100). If the rotation angle calculation unit 5063b determines that the rotation angle of the oblique-viewing endoscope 100 has not changed (NO in step S100), the rotation angle calculation unit 5063b repeats the processing of step S100.

On the other hand, when the rotation angle calculation unit 5063b determines that the rotation angle of the oblique-viewing endoscope 100 has changed (YES in step S100), a mark generation unit 5063f generates the mark M102 corresponding to a rotation angle α of the oblique-viewing endoscope 100, and outputs the mark M102 to an image processing unit 5061. As a result, the image processing unit 5061 generates an image on which the mark M102 is superimposed, and a display device 5041 displays the image on which the mark M100 indicating the gravity direction is superimposed (step S302).

Next, a direction generation unit 5063e determines whether or not the camera head 5005 has rotated by using the output signal of an acceleration sensor 5015 (step S304). If the direction generation unit 5063e determines that the camera head 5005 has not rotated (NO in step S304), the direction generation unit 5063e repeats the processing of step S304.

On the other hand, if the direction generation unit 5063e determines that the rotation angle of the camera head 5005 has changed (YES in step S304), the direction generation unit 5063e calculates a rotation angle γ of the camera head 5005 from the gravity direction (step S306), and the mark generation unit 5063f generates the mark M102 corresponding to the rotation angle α of the oblique-viewing endoscope 100 and the rotation angle γ again and outputs the mark M102 to the image processing unit 5061. As a result, the image processing unit 5061 generates an image on which the new mark M102 is superimposed, and the display device 5041 displays the image on which the mark M102 indicating the gravity direction is superimposed (step S308).

As described above, in the endoscope unit 5006 according to the present embodiment, the camera head 5005 is manually rotatable and controllable by the operator. In this case, the mark generation unit 5063f generates the mark M102 corresponding to the rotation angle α of the oblique-viewing endoscope 100, and outputs the mark M102 to the image processing unit 5061. As a result, the image processing unit 5061 generates an image on which the mark M102 is superimposed, and the display device 5041 displays the image on which the mark M100 indicating the gravity direction is superimposed. As a result, the operator can rotate the camera head 5005 with respect to the holding unit portion 7000 by the rotation angle γ according to the direction indicated by the mark M102. Then, when the camera head 5005 is rotated by the operator, the mark generation unit 5063f generates the mark M102 corresponding to the rotation angle α of the oblique-viewing endoscope 100 and the rotation angle γ again and outputs the mark M102 to the image processing unit 5061. As a result, the image processing unit 5061 generates an image on which the new mark M102 is superimposed, and the display device 5041 displays the image on which the mark M102 indicating the new rotation direction is superimposed again. At this time, since the length of the mark M102 changes, the operator can appropriately control the top and bottom of the image by rotating the camera head 5005 while confirming the direction of the camera head 5005 rotated by the operator.

### Modification 2 of Second Embodiment

An endoscope unit 5006d according to Modification 2 of the second embodiment is different from the endoscope unit 5006 according to the second embodiment in that a camera head 5005a and a holding unit portion 7000a are integrally formed. Hereinafter, differences from the endoscope unit 5006 according to the second embodiment will be described.

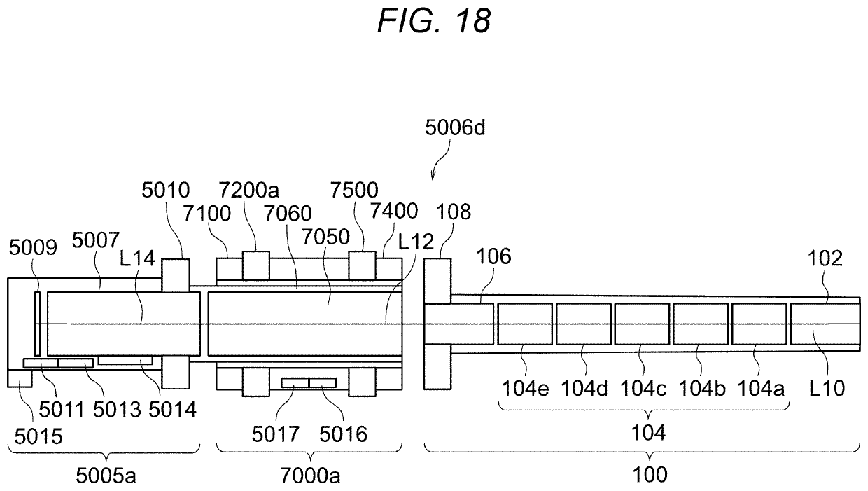

FIG. 18 is a diagram illustrating a configuration example of the endoscope unit 5006d according to Modification 2 of the second embodiment. As illustrated in FIG. 18, the camera head 5005a and the holding unit portion 7000a are integrally formed. Note that the camera head 5005 and the holding unit portion 7000 may also be integrally formed in the endoscope unit 5006 according to the first embodiment. In this manner, by integrally forming the camera head 5005a and the holding unit portion 7000a, airtightness can be further enhanced.

### Modification 3 of Second Embodiment

An endoscope unit 5006e according to Modification 3 of the second embodiment is different from the endoscope unit 5006 according to the second embodiment in that a camera head 5005b is formed integrally with a camera head 5005, a second relay optical system 7050, and a housing 7060 according to the first embodiment. Hereinafter, differences from the endoscope unit 5006 according to the second embodiment will be described.

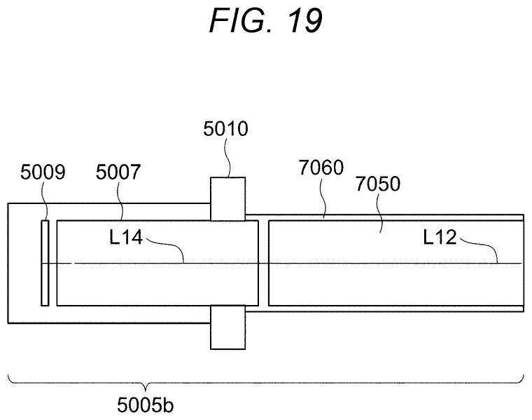

FIG. 19 is a diagram illustrating a configuration example of the camera head 5005b according to Modification 3 of the second embodiment. As illustrated in FIG. 19, the camera head 5005, the second relay optical system 7050, and the housing 7060 are integrally formed. Furthermore, in FIG. 19, only the configuration of the camera head 5005b is illustrated in order to simplify the description.

The camera head 5005, the second relay optical system 7050, and the housing 7060 may also be integrally formed in the endoscope unit 5006 according to the first embodiment. In this manner, by integrally forming the camera head 5005, the second relay optical system 7050, and the housing 7060, airtightness can be further enhanced.

### Third Embodiment

An endoscope unit 5006e according to a third embodiment is different from the endoscope unit 5006d according to Modification 2 of the second embodiment in that a forward-viewing endoscope 100a is provided instead of the oblique-viewing endoscope 100. Hereinafter, differences from the endoscope unit 5006d according to Modification 2 of the second embodiment will be described.

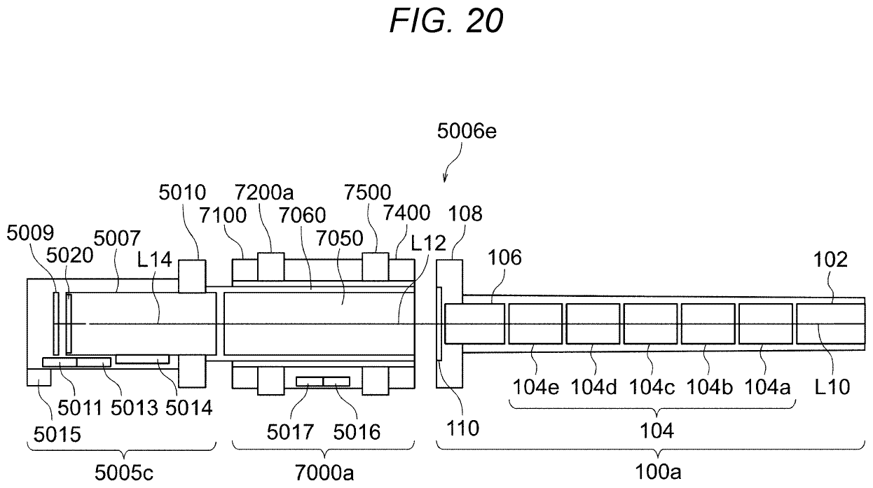

FIG. 20 is a diagram illustrating a configuration example of the endoscope unit 5006e according to the third embodiment. As illustrated in FIG. 20, the endoscope unit 5006e is different from the endoscope unit 5006d according to Modification 2 of the second embodiment in that a forward-viewing endoscope 100a is provided instead of the oblique-viewing endoscope 100. In addition, a first polarizing filter 110 is provided at an end portion on the output side of the forward-viewing endoscope 100a. Similarly, a second polarizing filter 5015 is provided at the front portion of an imaging unit 5009 of a camera head 5005c. As a result, the endoscope unit 5006e according to the third embodiment can perform imaging subjected to polarization processing.

On the other hand, if the orientations of the first polarizing filter 110 and the second polarizing filter 5015 do not match a predetermined orientation, appropriate polarization processing is not performed. Therefore, in the endoscope unit 5006e according to the third embodiment, rotation control of the camera head 5005c and the forward-viewing endoscope 100a equivalent to that described in the first embodiment to Modification 2 of the second embodiment is performed. As a result, even in a case where the forward-viewing endoscope 100a is rotated, the orientations of the first polarizing filter 110 and the second polarizing filter 5015 can be matched with a predetermined direction.

Note that the present technology can also have the following configurations.

(1)

An endoscope holding device including:
a housing that accommodates a relay optical system and is disposed in a robot arm;
a first rotation unit that is disposed in the housing and rotates an endoscope with respect to an optical axis of the relay optical system, the endoscope being fixed such that light emitted from the endoscope enters the relay optical system; and
a second rotation unit that is disposed in the housing and rotates an imaging device with respect to the optical axis of the relay optical system, the imaging device being fixed such that light emitted from the relay optical system enters the imaging device.

(2)

The endoscope holding device according to (1), further including a first attachment portion that detachably attaches the endoscope to the first rotation unit.

(3)

The endoscope holding device according to (2), in which the first attachment portion fixes the endoscope such that a first optical axis at a subsequent stage of the endoscope coincides with a second optical axis of the relay optical system.

(4)

The endoscope holding device according to (3), further including a first rotation angle acquisition unit that acquires a rotation angle of the endoscope rotating around the first optical axis.

(5)

The endoscope holding device according to (4), further including a first electric motor unit that rotates the endoscope around the first optical axis.

(6)

The endoscope holding device according to (4), in which the second optical axis and a third optical axis of an optical system of the imaging device are fixed to coincide with each other.

(7)

The endoscope holding device according to (6), in which the second rotation unit rotates the imaging device around the third optical axis on the basis of information of a rotation angle of the endoscope acquired by the first rotation angle acquisition unit.

(8)

The endoscope holding device according to (7), further including a second attachment portion that detachably attaches the imaging device to the second rotation unit.

(9)

The endoscope holding device according to (7), in which the relay optical system and an optical system of the imaging device are integrally formed.

(10)

The endoscope holding device according to (7), further including a second electric motor unit that rotates the imaging device around the third optical axis.

(11)

The endoscope holding device according to (10), in which the second electric motor unit rotates the imaging device around the third optical axis on the basis of information of a rotation angle of the endoscope.

(12)

The endoscope holding device according to (11), in which the endoscope is an oblique-viewing endoscope.

(13)

The endoscope holding device according to (12), in which the second electric motor unit rotates the imaging device with respect to the third optical axis on the basis of information of a rotation angle of the endoscope such that the imaging device maintains a predetermined rotation angle with respect to the third optical axis.

(14)

The endoscope holding device according to (10), in which the endoscope is a forward-viewing endoscope.

(15)

The endoscope holding device according to (14), in which
a first polarizing filter is disposed in an optical system of the endoscope, and a second polarizing filter is disposed in an optical system of the imaging device, and
the second electric motor unit rotates the imaging device with respect to the third optical axis on the basis of information of a rotation angle of the endoscope such that the first polarizing filter and the second polarizing filter maintain a predetermined rotation angle.

(16)

An endoscopic surgery system including:
the endoscope holding device according to (4);
a mark generation unit that generates a mark indicating at least one of a predetermined direction with respect to a third optical axis of the imaging device and a rotation direction by using information of a rotation angle of the endoscope acquired by the first rotation angle acquisition unit; and
a control unit that causes a display device to display an image captured by the imaging device with the mark superimposed on the image.

(17)

The endoscopic surgery system according to (16), in which
an acceleration sensor is disposed in the imaging device, and
the mark generation unit generates a mark indicating at least one of a predetermined direction with respect to the third optical axis and a rotation direction by also using information of the acceleration sensor.

(18)

The endoscopic surgery system according to (17), in which the mark generation unit generates a mark indicating information of the rotation direction and an angle to be rotated.

(19)

The endoscopic surgery system according to (18), further including a support arm device that supports the robot arm including the endoscope holding device at an end portion.

(20)

A control method of an endoscope holding device including
a first rotation step of rotating an endoscope with respect to an optical axis of a relay optical system, the endoscope being fixed to one end of a housing accommodating the relay optical system such that light emitted from the endoscope enters the relay optical system, and
a second rotation step of rotating an imaging device with respect to the optical axis of the relay optical system according to a rotation angle of the endoscope, the imaging device being fixed to the other end of the housing such that light emitted from the relay optical system enters the imaging device.

Aspects of the present disclosure are not limited to the above-described individual embodiments, but include various modifications that can be conceived by those skilled in the art, and the effects of the present disclosure are not limited to the above-described contents. That is, various additions, modifications, and partial deletions are possible without departing from the conceptual idea and spirit of the present disclosure derived from the matters defined in the claims and equivalents thereof.

### REFERENCE SIGNS LIST

100 Oblique-viewing endoscope (endoscope)
100a Forward-viewing endoscope (endoscope)
1350 Arm control device
1420 Robot arm
5000 Endoscopic surgery system
5005 Camera head (imaging device)
5041 Display device
5063a Rotation angle acquisition unit
5063f Mark generation unit
7000 Holding unit portion (endoscope holding device)
7050 Second relay optical system (relay optical system)
7060 Housing
7100 Camera head attachment portion (second attachment portion)
7200 Camera head rotation drive unit (second rotation unit)
7210 Actuator (second electric motor unit)
7400 Endoscope attachment portion (first attachment portion)
7500 Oblique-viewing endoscope rotation drive unit (first rotation unit)
7510 Actuator (first electric motor unit)
M100, M102 Mark

1. An endoscope holding device comprising:
a housing that accommodates a relay optical system and is disposed in a robot arm;
a first rotation unit that is disposed in the housing and rotates an endoscope with respect to an optical axis of the relay optical system, the endoscope being fixed such that light emitted from the endoscope enters the relay optical system; and
a second rotation unit that is disposed in the housing and rotates an imaging device with respect to the optical axis of the relay optical system, the imaging device being fixed such that light emitted from the relay optical system enters the imaging device.

2. The endoscope holding device according to claim 1, further comprising a first attachment portion that detachably attaches the endoscope to the first rotation unit.

3. The endoscope holding device according to claim 2, wherein the first attachment portion fixes the endoscope such that a first optical axis at a subsequent stage of the endoscope coincides with a second optical axis of the relay optical system.

4. The endoscope holding device according to claim 3, further comprising a first rotation angle acquisition unit that acquires a rotation angle of the endoscope rotating around the first optical axis.

5. The endoscope holding device according to claim 4, further comprising a first electric motor unit that rotates the endoscope around the first optical axis.

6. The endoscope holding device according to claim 4, wherein the second optical axis and a third optical axis of an optical system of the imaging device are fixed to coincide with each other.

7. The endoscope holding device according to claim 6, wherein the second rotation unit rotates the imaging device around the third optical axis on a basis of information of a rotation angle of the endoscope acquired by the first rotation angle acquisition unit.

8. The endoscope holding device according to claim 7, further comprising a second attachment portion that detachably attaches the imaging device to the second rotation unit.

9. The endoscope holding device according to claim 7, wherein the relay optical system and an optical system of the imaging device are integrally formed.

10. The endoscope holding device according to claim 7, further comprising a second electric motor unit that rotates the imaging device around the third optical axis.

11. The endoscope holding device according to claim 10, wherein the second electric motor unit rotates the imaging device around the third optical axis on a basis of information of a rotation angle of the endoscope.

12. The endoscope holding device according to claim 11, wherein the endoscope is an oblique-viewing endoscope.

13. The endoscope holding device according to claim 12, wherein the second electric motor unit rotates the imaging device with respect to the third optical axis on a basis of information of a rotation angle of the endoscope such that the imaging device maintains a predetermined rotation angle with respect to the third optical axis.

14. The endoscope holding device according to claim 10, wherein the endoscope is a forward-viewing endoscope.

15. The endoscope holding device according to claim 14, wherein
a first polarizing filter is disposed in an optical system of the endoscope, and a second polarizing filter is disposed in an optical system of the imaging device, and
the second electric motor unit rotates the imaging device with respect to the third optical axis on a basis of information of a rotation angle of the endoscope such that the first polarizing filter and the second polarizing filter maintain a predetermined rotation angle.

16. An endoscopic surgery system comprising:
the endoscope holding device according to claim 4;
a mark generation unit that generates a mark indicating at least one of a predetermined direction with respect to a third optical axis of the imaging device and a rotation direction by using information of a rotation angle of the endoscope acquired by the first rotation angle acquisition unit; and
a control unit that causes a display device to display an image captured by the imaging device with the mark superimposed on the image.

17. The endoscopic surgery system according to claim 16, wherein
an acceleration sensor is disposed in the imaging device, and
the mark generation unit generates a mark indicating at least one of a predetermined direction with respect to the third optical axis and a rotation direction by also using information of the acceleration sensor.

18. The endoscopic surgery system according to claim 17, wherein the mark generation unit generates a mark indicating information of the rotation direction and an angle to be rotated.

19. The endoscopic surgery system according to claim 18, further comprising a support arm device that supports the robot arm including the endoscope holding device at an end portion.

20. A control method of an endoscope holding device comprising
a first rotation step of rotating an endoscope with respect to an optical axis of a relay optical system, the endoscope being fixed to one end of a housing accommodating the relay optical system such that light emitted from the endoscope enters the relay optical system, and
a second rotation step of rotating an imaging device with respect to the optical axis of the relay optical system according to a rotation angle of the endoscope, the imaging device being fixed to the other end of the housing such that light emitted from the relay optical system enters the imaging device.
